(19) United States
(12) Patent Application Publication
(10) Pub. No.: US 2007/0254895 A1
(43) Pub. Date: Nov. 1, 2007

(54) Crystalline solvate of glucokinase activator

(76) Inventors: Duk Soon Choi, Flanders, NJ (US); Hitesh Parmendra Chokshi, Clifton, NJ (US); Ahmad Waseem Malick, Edison, NJ (US); Roumen Nikolaev Radinov, West Caldwell, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 11/786,309

(22) Filed: Apr. 11, 2007

Related U.S. Application Data
(60) Provisional application No. 60/791,255, filed on Apr. 12, 2006.

(51) Int. Cl. A61K 31/4965

(52) U.S. Cl. 514/255.06; 544/406

(57) ABSTRACT

Provided is a crystalline IPA solvate of 2(R)-(3-Chloro-4-methanesulfonyl-phenyl)-3-((R)-3-oxo-cyclopentyl)-N-pyrazin-2-yl-propionamide as a glucokinase activator which increases insulin secretion in the treatment of, for example, type II diabetes.

### PRIORITY TO RELATED APPLICATIONS

This application claims the benefit of U.S. Provisional Application No. 60/791,255, filed Apr. 12, 2006, which is hereby incorporated by reference in its entirety.

### FIELD OF THE INVENTION

The invention is directed to the crystalline isopropanol (IPA) solvate of 2(R)-(3-Chloro-4-methanesulfonyl-phenyl)-3-((R)-3-oxo-cyclopentyl)-N-pyrazin-2-yl-propionamide of the formula (I):

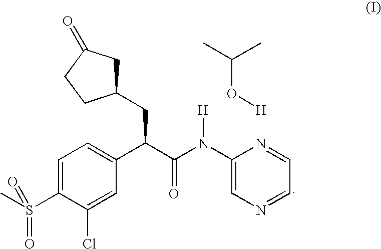

The invention is also directed to pharmaceutical compositions comprising (R)-(3-Chloro-4-methanesulfonyl-phenyl)-3-((R)-3-oxo-cyclopentyl)-N-pyrazin-2-yl-propionamide, as well as methods directed to its use as a glucokinase activator for the treatment of metabolic diseases and disorders.

All documents cited or relied upon herein are expressly incorporated herein by reference.

### BACKGROUND OF THE INVENTION

Glucokinase (GK) is one of four hexokinases that are found in mammals [Colowick, S. P., in The Enzymes, Vol. 9 (P. Boyer, ed.) Academic Press, New York, N.Y., pages 1-48, 1973]. The hexokinases catalyze the first step in the metabolism of glucose, i.e., the conversion of glucose to glucose-6-phosphate. Glucokinase has a limited cellular distribution, being found principally in pancreatic 3-cells and liver parenchymal cells. In addition, GK is a rate-controlling enzyme for glucose metabolism in these two cell types that are known to play critical roles in whole-body glucose homeostasis [Chipkin, S. R., Kelly, K. L., and Ruderman, N. B. in Joslin's Diabetes (C. R. Khan and G. C. Wier, eds.), Lea and Febiger, Philadelphia, Pa., pages 97-115, 1994]. The concentration of glucose at which GK demonstrates half-maximal activity is approximately 8 mM. The other three hexokinases are saturated with glucose at much lower concentrations (<1 mM). Therefore, the flux of glucose through the GK pathway rises as the concentration of glucose in the blood increases from fasting (5 mM) to postprandial (≈10-15 mM) levels following a carbohydrate-containing meal [Printz, R. G., Magnuson, M. A., and Granner, D. K. in Ann. Rev. Nutrition Vol. 13 (R. E. Olson, D. M. Bier, and D. B. McCormick, eds.), Annual Review, Inc., Palo Alto, Calif., pages 463-496, 1993]. These findings contributed over a decade ago to the hypothesis that GK functions as a glucose sensor in β-cells and hepatocytes (Meglasson, M. D. and Matschinsky, F. M. Amer. J. Physiol. 246, E1-E13, 1984). In recent years, studies in transgenic animals have confirmed that GK does indeed play a critical role in whole-body glucose homeostasis. Animals that do not express GK die within days of birth with severe diabetes while animals overexpressing GK have improved glucose tolerance (Grupe, A., Hultgren, B., Ryan, A. et al., Cell 83, 69-78, 1995; Ferrie, T., Riu, E., Bosch, F. et al., FASEB J, 10, 1213-1218, 1996). An increase in glucose exposure is coupled through GK in β-cells to increased insulin secretion and in hepatocytes to increased glycogen deposition and perhaps decreased glucose production.

The finding that type II maturity-onset diabetes of the young (MODY-2) is caused by loss of function mutations in the GK gene suggests that GK also functions as a glucose sensor in humans (Liang, Y., Kesavan, P., Wang, L. et al., Biochem. J 309, 167-173, 1995). Additional evidence supporting an important role for GK in the regulation of glucose metabolism in humans was provided by the identification of patients that express a mutant form of GK with increased enzymatic activity. These patients exhibit a fasting hypoglycemia associated with an inappropriately elevated level of plasma insulin (Glaser, B., Kesavan, P., Heyman, M. et al., New England J Med. 338, 226-230, 1998). While mutations of the GK gene are not found in the majority of patients with type II diabetes, compounds that activate GK, and thereby increase the sensitivity of the GK sensor system, will still be useful in the treatment of the hyperglycemia characteristic of all type II diabetes. Glucokinase activators will increase the flux of glucose metabolism in β-cells and hepatocytes, which will be coupled to increased insulin secretion. Such agents would be useful for treating type II diabetes.

### SUMMARY OF THE INVENTION

In an embodiment of the present invention, provided is a crystalline isopropanol solvate of 2(R)-(3-Chloro-4-methanesulfonyl-phenyl)-3-((R)-3-oxo-cyclopentyl)-N-pyrazin-2-yl-propionamide of the formula (I):

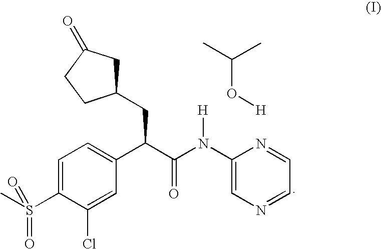

In another embodiment of the present invention, provided is a pharmaceutical composition, comprising a therapeutically effective amount of 2(R)-(3-Chloro-4-methanesulfonyl-phenyl)-3-((R)-3-oxo-cyclopentyl)-N-pyrazin-2-yl-propionamide, and a pharmaceutically acceptable carrier.

In a further embodiment of the present invention, provided is a method for the treatment of a metabolic disease or disorder, comprising administering a therapeutically effective amount of 2(R)-(3-Chloro-4-methanesulfonyl-phenyl)-3-((R)-3-oxo-cyclopentyl)-N-pyrazin-2-yl-propionamide to a patient in need thereof.

### BRIEF DESCRIPTION OF THE DRAWINGS

FIG. 1 shows the molecular structure of the IPA solvate of the formula (I).

FIG. 2 shows the molecular packing for 2(R)-(3-Chloro-4-methanesulfonyl-phenyl)-3-((R)-3-oxo-cyclopentyl)-N-pyrazin-2-yl-propionamide from the crystal structure.

FIG. 3 shows a photomicrograph of the IPA solvate of the formula (I).

FIG. 4 shows a photomicrograph of the IPA Solvate of the Formula (I) at 2000×.

FIG. 5 shows a powder XRD pattern of the IPA solvate of the formula (I) and amorphous form.

Figure shows a 6DSC thermogram of IPA solvate of the formula (I) and amorphous form.

Figure shows a 7TGA thermogram of IPA solvate of the formula (I) and amorphous form.

FIG. 8 shows a TGA-IR of IPA solvate of the formula (I).

FIG. 9 shows a DSC and TGA overlay of the IPA solvate of the formula (I).

FIG. 10 shows a TGA thermogram and derivatives.

FIG. 11 shows a TGA thermogram of IPA solvate of the formula (I).

FIG. 12 shows moisture sorption isotherms of IPA solvate of the formula (I) and amorphous form.

FIG. 13 shows moisture sorption isotherms of IPA solvate of the formula (I) at 25° C. and 40° C.

FIG. 14 shows moisture sorption isotherms of IPA solvate of the formula (I) with different particle size distributions.

FIG. 15 shows DSC thermograms of calibration samples.

FIG. 16 shows a plot of ΔH vs % crystallinity.

FIG. 17 shows a plot of heat of fusion against IPA level in IPA solvate of the formula (I).

FIG. 18. shows a powder XRD pattern of water vapor treated IPA solvate of the formula (I).

### DETAILED DESCRIPTION OF THE INVENTION

This invention provides, for example, a crystalline isopropanol (IPA) solvate of 2(R)-(3-Chloro-4-methanesulfonyl-phenyl)-3-((R)-3-oxo-cyclopentyl)-N-pyrazin-2-yl-propionamide of the formula (I):

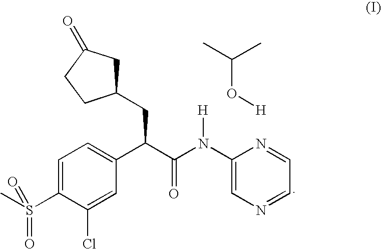

In a preferred embodiment, the compound of formula (I) is a crystalline mono isopropyl alcohol solvate with (R,R) configuration having one powder XRD pattern. The isopropyl alcohol is one important part of the crystalline lattice and the crystal structure collapses when alcohol is removed.

As described in the Examples below, the IPA solvate melted around 94° C. with desolvation. The evaporation of isopropyl alcohol from the melt was slow over the wide range above the melting temperature. The IPA solvate interacted with water vapor at different vapor pressures depending on temperature and apparent particle sizes. At higher temperature it interacted at a lower vapor pressure. At smaller particle size, it interacted at a lower vapor pressure. The interaction with water vapor resulted in the loss of isopropyl alcohol, which translated to the loss of crystallinity. Both heat of fusion and the level of isopropyl alcohol was used to estimate the crystallinity of the solvate. The IPA solvate was stable when protected from high humidity.

It is to be understood that the terminology employed herein is for the purpose of describing particular embodiments, and is not intended to be limiting. Further, although any methods, devices and materials similar or equivalent to those described herein can be used in the practice or testing of the invention, the preferred methods, devices and materials are now described.

As used herein, the term “alkyl” means, for example, a branched or unbranched, cyclic or acyclic, saturated or unsaturated (e.g. alkenyl or alkynyl) hydrocarbyl radical which may be substituted or unsubstituted. Where cyclic, the alkyl group is preferably C₃ to C₁₂, more preferably C₅ to C₁₀, more preferably C₅ to C₇. Where acyclic, the alkyl group is preferably C₁ to C₁₀, more preferably C₁ to C₆, more preferably methyl, ethyl, propyl (n-propyl or isopropyl), butyl (n-butyl, isobutyl, sec-butyl or tertiary-butyl) or pentyl (including n-pentyl and isopentyl), more preferably methyl. It will be appreciated therefore that the term “alkyl” as used herein includes alkyl (branched or unbranched), substituted alkyl (branched or unbranched), alkenyl (branched or unbranched), substituted alkenyl (branched or unbranched), alkynyl (branched or unbranched), substituted alkynyl (branched or unbranched), cycloalkyl, substituted cycloalkyl, cycloalkenyl, substituted cycloalkenyl, cycloalkynyl and substituted cycloalkynyl.

As used herein, the term “lower alkyl” means, for example, a branched or unbranched, cyclic or acyclic, saturated or unsaturated (e.g. alkenyl or alkynyl) hydrocarbyl radical wherein said cyclic lower alkyl group is C₃, C₄, C₅, C₆ or C₇, and wherein said acyclic lower alkyl group is C₁, C₂, C₃ or C₄, and is preferably selected from methyl, ethyl, propyl (n-propyl or isopropyl) or butyl (n-butyl, sec-butyl, isobutyl or tertiary-butyl). It will be appreciated therefore that the term “lower alkyl” as used herein includes lower alkyl (branched or unbranched), lower alkenyl (branched or unbranched), lower alkynyl (branched or unbranched), cycloloweralkyl, cycloloweralkenyl and cycloloweralkynyl.

The alkyl group may be substituted or unsubstituted. Where substituted, there will generally be, for example, 1 to 3 substituents present, preferably 1 substituent. Substituents may include, for example: carbon-containing groups such as alkyl, aryl and arylalkyl (e.g. substituted and unsubstituted phenyl, substituted and unsubstituted benzyl).

The lower alkyl groups may be substituted or unsubstituted, preferably unsubstituted. Where substituted, there will generally be, for example, 1 to 3 substitutents present, preferably 1 substituent.

In the practice of the method of the present invention, an effective amount of any one of the compounds of this invention or a combination of any of the compounds of this invention or a pharmaceutically acceptable salt or ester thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form or solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The pharmaceutical preparations can also contain preserving agents, solubilizing agents, stabilizing agents, wetting agents, emulsifying agents, sweetening agents, coloring agents, flavoring agents, salts for varying the osmotic pressure, buffers, coating agents or antioxidants. They can also contain other therapeutically valuable substances, including additional active ingredients.

The “therapeutically effective amount” or “dosage” of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 kg, a daily dosage of from about 0.01 mg/kg to about 50 mg/kg should be appropriate, although the upper limit may be exceeded when indicated. The dosage is preferably from about 0.3 mg/kg to about 10 mg/kg per day. A preferred dosage may be from about 0.70 mg/kg to about 3.5 mg/kg per day. The daily dosage can be administered as a single dose or in divided doses, or for parenteral administration it may be given as continuous infusion.

The compounds of the present invention can be prepared by any conventional manner. Suitable processes for synthesizing these compounds are provided in the examples. Generally, the compounds can be prepared according to the Reaction Scheme described below. The sources of the starting materials for these reactions are also described.

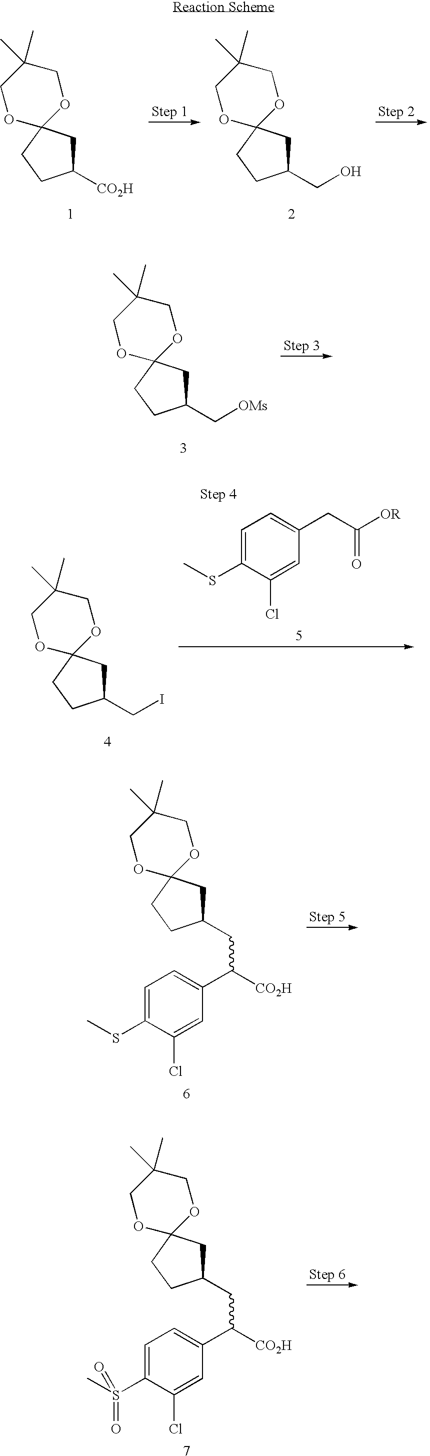

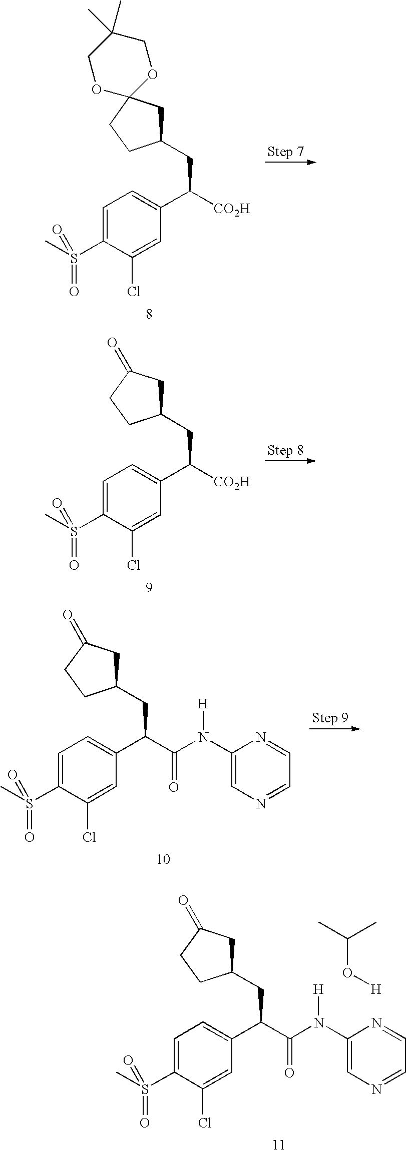

The starting materials, (S)-ketal acid 1 and ester 5, were prepared according to known methods (e.g., DE 4312832C1 for the acid 1). An acyclic or cyclic ketal protective group may be used for the (S)-ketal acid of the formula:

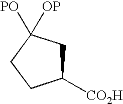

where P is an alkyl group or P—P is an alkylidene group forming an acyclic or cyclic ketal protective group, such as an acyclic dialkyl ketal or a cyclic unsubstituted or substituted 1,3-dioxolane or 1,3-dioxane, or other carbonyl protective group. The protective group is introduced using conventional procedures, e.g. by treating the keto acid with an alcohol or diol in the presence of acid. However, the cyclic ketal 5,5,-dimethyl-1,3-dioxolane is a preferred protective group for (S)-ketal acid 1.

In Step 1 of the Reaction Scheme, (S)-ketal acid 1 or its salts can be used alternatively. If an amine salt of the acid is used, the free acid can be obtained from the salts by known methods. For example, the amine salt of 1 was treated with a citric acid solution then the free acid 1 was extracted with toluene and the solvent was removed by vacuum distillation.

Acid 1 was converted to iodide 4 by standard procedures. Thus, alcohol 2 was obtained from acid 1 by reduction. For example, addition of a THF solution of lithium aluminum hydride (LAH) to a toluene solution of 1 produced alcohol 2. Alcohol 2 was converted to the iodide 4 via an activated ester such as the mesylate 3. Mesylate 3 was obtained from alcohol by reaction with metanesulfonyl chloride and a base, e.g. DABCO, and then converted to iodide 4 by reaction with an iodide salt, e.g. sodium iodide, in the presence of an amine, e.g. diisopropylethylamine. The iodide 4 can be also obtained using other methods.

As described above for the (S)-ketal acid 1, an acyclic or cyclic ketal protective group may be also used for the iodide of the formula:

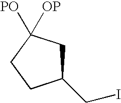

where P is an alkyl group or P—P is an alkylidene group forming an acyclic or cyclic ketal protective group, such as an acyclic dialkyl ketal or a cyclic unsubstituted or substituted 1,3-dioxolane or 1,3-dioxane, or other carbonyl protective group, However, the cyclic ketal 5,5,-dimethyl-1,3-dioxolane is preferred as the protective group for the iodide 4.

In Step 4, deprotonation of ethyl ester 5, followed by the addition of iodide 4 and DMPU gave the corresponding ethyl ester of 6, which was hydrolyzed in situ by the addition of aqueous sodium hydroxide and methanol to provide acid 6. Various bases can be used for the deprotonation of esters such as 5, e.g. LDA, LiHMDS, NaHMDS, KHMDS. However, LiHMDS in THF is preferred.

The resulting sulfide 6 was oxidized to the sulfone 7. Various methods can be used for oxidation, such as DMDO, Oxone® or hydrogen peroxide. In the preferred example, tungstate-catalyzed oxidation with hydrogen peroxide provided the sulfone 7 which was conveniently isolated as a salt with an amine, e.g. α-methylbenzylamine or dicyclohexylamine (Step 5). While 7 could be used directly without isolation, its isolation as a salt is preferred because this additional purification results in better yield and purity for the next step.

As described above for the (S)-ketal acid 1, an acyclic or cyclic ketal protective group may be also used for the acid of formula

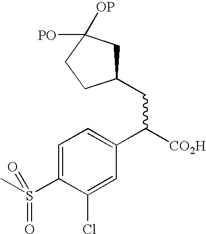

where P is an alkyl group or P—P is an alkylidene group forming an acyclic or cyclic ketal protective group, such as an acyclic dialkyl ketal or a cyclic unsubstituted or substituted 1,3-dioxolane or 1,3-dioxane, or other carbonyl protective group, However, the cyclic ketal 5,5,-dimethyl-1,3-dioxolane is preferred as the protective group for the acid 7.

In Step 6, the acid 7 which is a mixture of epimers, can be converted to the single epimer 9 by treatment with base under such conditions that the desired epimer salt of 8 crystallizes out of solution while the undesired epimer salt remains in solution where it is converted to 8. It is preferable to use a sodium salt of 8 in an alcohol solvent such as ethanol. Thus acid 7 was converted to its sodium salt, e.g. by treatment with sodium tert-butoxide. After solvent exchange to ethanol, additional sodium tert-butoxide was added, and the suspension was concentrated and heated to reflux to accomplish the selective epimerization to 8 via crystallization-induced dynamic resolution. After cooling to room temperature, the desired sodium salt of 8 was isolated by filtration. Ketal deprotection of 8 using aqueous acid in acetone (Step 7) provided keto acid 9 which can be isolated by crystallization.

In Step 8, the coupling of 9 with 2-aminopyrazine provided amide 10 is as described in WO03/095438. After solvent exchange to isopropanol, the diastereomerically pure IPA solvate, 11, crystallized and was isolated by filtration.

Further details regarding the steps in the reaction scheme are as follows:

### Step 1. Preparation of Alcohol 2

(S)-Ketal acid 1 as its (S) α-methylbenzylamine salt with a chiral purity of 94% ee was used in this preparation. After acidification with citric acid, the free acid 1 was extracted with toluene. The toluene solution was concentrated to remove residual water. Then, addition of lithium aluminum hydride (0.87 mol-equiv.; 1.15 equiv. based on hydride) in THF at 50° C. cleanly produced alcohol 2. The reaction exotherm was controlled by the addition rate and external cooling. The reaction was quenched by the addition of concentrated ammonium hydroxide, followed by sodium sulfate, to provide an easily filterable solid. The salts were removed by filtration and the filtrates were partially concentrated. This workup procedure produced pure product, 2. A similar procedure using Red-A1® (1.5 equiv.; 1 equiv. based on hydride) also gave a clean conversion, but resulted in a lower isolated yield (85%) of 2. The concentrated toluene solution of crude 2 was diluted with ethyl acetate and used directly in Step 2.

### Step 2. Preparation of Mesylate 3

Addition of DABCO (1.8 equiv.) to the solution of 2 in ethyl acetate from the previous step, followed by the addition of methanesulfonyl chloride (1.5 equiv.) at 0° C. and warming to room temperature, produced mesylate 3. The reaction was quenched by the addition of water and the organic phase was separated and partially concentrated. After addition of diisopropylethylaamine (DIPEA), the concentrated toluene solution of 3 was diluted with acetone and used directly in Step 3.

### Step 3. Preparation of Iodide 4

To the solution of mesylate 3 in acetone from the previous step was added DIPEA (a total of ca. 1.9 equiv.), followed by sodium iodide (3.7 equiv.) in this order, and the mixture was heated to reflux for 15 h to provide iodide 4. The addition order of the above reagents is an important factor. A complex mixture was obtained without DIPEA or when using inorganic bases (i.e., sodium bicarbonate or sodium carbonate). The reaction mixture was diluted with aqueous potassium bicarbonate and was partially concentrated to remove acetone. Then, the product was extracted with heptane and the organic phase was washed with water and concentrated. Crude iodide 4, thus obtained, was used directly for the alkylation Step 4.

### Step 4. Preparation of Sulfide Acid 6

While the corresponding acid or its esters can also be used for alkylation, the ethyl ester is preferred. The ethyl ester 5 was deprotonated by the addition of LiHMDS (1.05 equiv.) in THF at −5° C., followed by stirring for at least 1 h. Then, a toluene solution of iodide 4 (1.03 equiv.) was added to the enolate (no exotherm), followed by 1.5 equiv. of DMPU (exotherm to 12° C.). The reaction mixture was allowed to stir at 20-22° C. for 16 h to achieve complete reaction (>90% conversion after 4-5 h). DMPU was added after complete enolate formation since the deprotonation of 5 with LiHMDS was cleaner in its absence. The formation of a bis alkylation byproduct is minimized following this procedure.

Hydrolysis of the alkylated ester was accomplished in one-pot by the addition of 2M sodium hydroxide (1.2 equiv.) and methanol and heating the mixture to 50° C. for 16 h, providing the acid 6.

After complete hydrolysis to 6, the mixture was concentrated and the resulting aqueous solution was washed with 1:1 heptane-ethyl acetate to remove non-acidic by-products, then acidified to pH 3-4 with citric acid and extracted with ethyl acetate. The organic extract was immediately mixed with aqueous bicarbonate solution to give a two-phase mixture; the pH of the aqueous phase was 7.5-8. This mixture was concentrated to remove the organic solvent, and the resulting aqueous solution of 10 was diluted with acetone and used directly in Step 5.

### Step 5. Preparation of Sulfone Acid 7

While various oxidation methods can be used, such as DMDO, oxone, hydrogen peroxide, etc. the following procedure is preferred. To an aqueous acetone solution of 6, prepared in Step 4, was added 5-15 mol % of sodium tungstate, and the pH of the mixture was adjusted to 8.0±0.3 before addition of the hydrogen peroxide. Deionized, chloride-free water was used for this preparation to prevent the formation of chlorinated by-products during oxidation. Hydrogen peroxide was then added to the reaction while maintaining a pH 7.5-8.0 until a complete conversion to the sulfone 7 was achieved.

After complete oxidation, as determined by HPLC analysis, excess peroxide was quenched by the addition of a sulfite and the pH was adjusted to >9. The mixture was concentrated under reduced pressure to remove acetone. The resulting aqueous solution of 7 was acidified to pH 3-4 by the addition of citric acid and extracted with ethyl acetate. Racemic a-methylbenzylamine (rac-MBA) was then added to the organic solution and, after solvent exchange to acetonitrile, the resulting MBA salt of 7, was isolated by filtration.

### Step 6. Preparation of Chiral Sodium Salt of Acid 8

The mixture of epimers 7 was converted to the desired epimer 8 by crystallization-induced dynamic resolution of the sodium salts. As the desired (2R,3′R) sodium salt of 8 crystallized preferentially from an ethanol solution, stereoselective epimerization was achieved by heating a concentrated ethanol solution of 7 to reflux in the presence of excess sodium alkoxide. Thereby, the desired (2R,3′R)-isomer 8 crystallized out as the sodium salt, while the (2S,3′R)-isomer remaining in solution gradually epimerized to 8.

Conversion of the MBA salt of 7 to free-acid 7 was accomplished by treatment with aqueous citric acid solution, followed by extraction with ethyl acetate. The ethyl acetate extract was washed with water containing 0.1 equiv. of sodium bicarbonate, increasing the purity of 7 from ca. 93 area % to >99 area %. After addition of 1 equiv. of sodium tert-butoxide, the solvent was exchanged to heptane, then to absolute ethanol by atmospheric distillation in order to remove ethyl acetate and reduce the water content to less than 0.3% (as determined by Karl-Fisher analysis). An additional 0.5 equiv. of sodium tert-butoxide was then added and the suspension was concentrated to 3-4 vol. and heated to reflux for 3.5 h to accomplish the selective epimerization to 8. After cooling to room temperature, a first crop of the desired sodium salt of 8 was isolated by filtration in 68.4% yield. Its chemical purity was 98.1% (disregarding diastereomers) and the diastereomeric ratio of the desired (2R,3′R)-isomer to the undesired isomers (2R,3′S and 2S,3′R, respectively) was 95.76:0.33:3.90 as determined by HPLC analysis. After concentration of the mother liquor, a second crop of the sodium salt of 8 was obtained in 14.5% yield, with a chemical purity of 97.0% and a diastereomeric ratio of 92.43:1.68:5.89. These two crops were separately subjected to the Step 7 ketal deprotection.

### Step 7. Preparation of Keto-Acid 9

Ketal deprotection of 8 using aqueous HCl in acetone provided crystalline keto-acid 9, which was isolated by filtration and recrystallized from acetone-heptane. Following this protocol, the first crop of 8, prepared above as the sodium salt, gave keto-acid 9 in 92% yield with a diastereomeric excess of 98.9%. For the second crop of 8, an additional recrystallization of the crude product from aqueous acetone was required to obtain 9 in a similar purity (diastereomeric excess of 99.6%) in 53% yield. These two lots were combined, giving a 71% overall yield of 9 from sulfone acid salt 7.

### Step 8. Preparation of IPA Solvate 11

The acid chloride coupling with aminopyrazine, using pyridine as the base, was used to convert acid 9 to amide 10. A dichloromethane solution of the corresponding acid chloride was generated from 9 by treatment with 1.05 equiv. of oxalyl chloride in the presence of a catalytic amount of DMF (6 mol %) at 20° C., partially concentrated under reduced pressure to remove residual hydrogen chloride and then added to a suspension of aminopyrazine (1.2 equiv.) and pyridine (1.5 equiv.) in dichloromethane at −15° C.. After allowing the temperature to rise to −5° C., the reaction mixture was quenched by the addition of water (2 equiv.) and silica gel (2.5 g per 1 g of 9) was added. After stirring the suspension for 1.5 h, the solids were removed by filtration and washed with 1:1 ethyl acetate-dichloromethane. This silica-gel treatment removed most of the colored by-products generated in the coupling reaction, including the oxalamide derived from the reaction of oxalyl chloride with aminopyrazine. At this stage, HPLC analysis indicated that the crude product 10 was 95.15% pure; the major contaminant was starting material 9 (4.45%). The combined filtrate and washes were concentrated, then washed successively with 1N hydrochloric acid to remove pyridine, 1M potassium bicarbonate solution to remove 9, and water. After solvent exchange to isopropanol, diastereomerically pure IPA solvate 11 crystallized from the mixture and was isolated by filtration in 81% yield from 9.

### EXAMPLES

### Example 1

### Preparation of Ester 5

A 500 mL flask equipped with a magnetic stirrer, Dean-Stark trap and reflux condenser was charged with 100 g (461.5 mmol) of 3-chloro-4-methylthiophenylacetic acid, 200 mL of ethanol and 4 mL (72 mmol) of concentrated sulfuric acid. After heating to 75° C. for 1 h, 100 mL of heptane was added and volatiles (ca. 120 mL) were removed by atmospheric distillation. Then, 50 mL of heptane was added and an additional 60 mL of volatiles were removed by distillation. In a similar manner, a total of 400 ML of 1:1 heptane:ethanol was added over the course of ca. 8 h, and an equivalent volume of distillate was collected. At this point, the temperature of the mixture was 84° C. and HPLC analysis indicated essentially complete reaction. After cooling to ambient temperature, the mixture (ca. 300 mL) was poured into a separatory funnel containing 100 mL of deionized water, 100 mL of ethyl acetate and 180 mL of heptane. After the two-phase mixture was thoroughly mixed, the organic layer was separated, washed with 50 mL of deionized water, followed by 50 mL (50 mmol) of 1M potassium bicarbonate, and concentrated under reduced pressure. The residue was diluted with 200 mL of heptane and the resulting solution was reconcentrated under reduced pressure to give 108.6 g (96.2% yield) of 5 as a light brown oil; 99.87% pure as determined by HPLC analysis.

### Example 2

### Preparation of Alcohol 2

A 500-mL separatory fimel was charged with 200 mL of toluene and 27.75 g (82.7 mmol) of the (S) α-methylbenzylamine salt of 1. Then, 114 mL (114 mmol) of 1M aqueous citric acid solution was added and the resulting heterogeneous mixture was thoroughly mixed. The organic layer was separated and the aqueous phase was back-extracted with 75 mL of toluene. The combined organic layers were concentrated at 45-50° C./52 torr to a weight of ca. 32 g. This clear, colorless solution was charged into a 250-mL, three-necked flask (equipped with a mechanical stirrer, thermometer, dropping fimel and nitrogen gas inlet/bubbler) and diluted with 52 mL of toluene. Then, 72 mL (72 mmol) of 1M lithium aluminum hydride in THF was added over 50 min. During the addition, the temperature of the reaction mixture was initially allowed to rise to 50° C. as a result of a reaction exotherm, then maintained at 50±3° C. by careful control of the addition rate. The dropping funnel was rinsed with a total of 10 mL of THF and the rinse was added to the mixture. The mixture was then stirred for 3.5 h without external heating. The reaction mixture was cooled with an ice-water bath and quenched by the addition of 9.3 mL (140 mmol) of concentrated ammonium hydroxide over 4 min, which caused gas evolution and an exotherm to 17° C.. The resulting mixture, containing a solid foam, was stirred for 5 min with ice-water cooling and 24.4 mL of 20% aqueous sodium sulfate was added over 1 min. The mixture was stirred for 10 min, then allowed to warm to ambient temperature over 30 min. The resulting suspension was filtered though a pad of Celite®. The filter aid and collected solids were washed with a total of 111 mL of THF. The combined filtrate and washes were concentrated at 40-45° C./80 torr to ca. half the original volume. The resulting concentrated solution was diluted with 200 mL of ethyl acetate and re-concentrated at 40-45° C./80 torr to a weight of ca. 21 g. The residue was diluted with 150 mL of ethyl acetate and the resulting solution of 2 was used directly in the next step.

### Example 3

### Preparation of Mesylate 3

A 500 mL, three-necked flask equipped with a mechanical stirrer, thermometer, dropping fimel and nitrogen gas inlet/bubbler was charged with 16.68 g (149 mmol) of DABCO™ and the ethyl acetate solution of 2 (ca. 170 mL) from the previous step, which was calculated to contain 16.57 g (82.7 mmol) of 2 and 150 mL of ethyl acetate. The resulting solution was cooled to −18° C. and 9.77 mL (126 mmol) of methanesulfonyl chloride was added over 2 min. The dropping funnel was rinsed with 8 mL of ethyl acetate and the rinse was added to the mixture. An exotherm that ensued raised the temperature to 8° C.. The resulting suspension was stirred for 10 min, then allowed to warm to ambient temperature over 3 h. TLC analysis indicated complete reaction. After 77 mL of deionized water was added, the mixture was stirred for 10 min, then diluted with 40 mL of toluene to facilitate phase separation. The organic layer was separated, washed with 2×40 mL=80 mL of deionized water, and concentrated at 42-46° C./80 torr. Then, 200 mL of ethyl acetate was added and the mixture was concentrated as described above to a weight of ca. 35 g. To this residue was added 8.3 mL (47.6 mmol) of DIPEA and 170 mL of acetone and the mixture was concentrated at 42-46° C./80 torr to a weight of ca. 28 g. This material was diluted with 220 mL of acetone and the resulting solution of 3 was used directly in the next step.

### Example 4

### Preparation of Iodide 4

A 500 mL, three-necked flask equipped with a mechanical stirrer, thermometer, condenser and nitrogen gas inlet/bubbler was charged with the acetone solution of 3 (ca. 250 mL) from the previous step, which was calculated to contain 23.03 g (82.7 mmol) of 3, ca. 8 mL of DIPEA and 220 mL of acetone. To the resulting solution was added 18.8 mL (108 mmol) of DIPEA and, after 5 min of stirring, 45.4 g (303 mmol) of sodium iodide was added. The mixture was stirred at room temperature for 15 min, then heated to 51° C. for 15.5 h. TLC analysis indicated complete reaction. After cooling to room temperature, 142 mL (142 mmol) of 1M potassium bicarbonate solution was added and the resulting mixture was concentrated at 40° C./60 torr to remove the organic solvent. The resulting aqueous mixture was then extracted with 200 mL of heptane. The organic layer was washed with 90 mL of deionized water and concentrated at 45° C./60 torr. The residue was dissolved in 180 mL of heptane and the solution was concentrated at 45° C./60 torr. The residue was then dried under high vacuum to give 23.52 g of 18 as an oil.

### Example 5

### Preparation of Sulfide Acid 6

A 1-L, three-necked flask equipped with a mechanical stirrer, thermometer, dropping funnel and nitrogen gas inlet/bubbler was charged with 25.01 g (102 mmol) of 5 and 114 mL of anhydrous THF. After cooling to −5° C., 107 mL (107 mmol) of 1M lithium bis(trimethylsilyl)amide (LiHMDS) in THF was added over 22 min, while maintaining the temperature of the reaction mixture between −2° C. and −5° C.. The resulting light brown solution was stirred at −5° C. for 1.5 h and a solution of 32.65 g (105 mmol) of 4 in 32 mL of toluene was added over 3 min (essentially no exotherm), followed by 18.5 mL (153 mmol) of DMPU in one portion (an exotherm ensued that raised the temperature of the mixture to 12° C. within 5 min). The reaction mixture was stirred at 22° C. for 25 h. HPLC and TLC analyses indicated essentially complete reaction (1.06 area % of 5 by HPLC). Then, 62.4 mL (125 mmol) of 2N sodium hydroxide and 124 mL of methanol were added and the mixture was heated to 50° C. for 2 h. TLC analysis indicated complete hydrolysis to 6. After cooling to ambient temperature overnight, the mixture was concentrated at 45° C./60 torr to remove the organic solvents. The resulting aqueous solution was washed with 2×100 mL=200 mL of 1:1 heptane:ethyl acetate and the combined organic layers were back-extracted with 30 mL (30 mmol) of 1N sodium hydroxide. The aqueous layers were combined and 300 mL of ethyl acetate was added. Then, to the vigorously stirred two-phase mixture was added 73 mL (235 mmol) of 50% aqueous citric acid, resulting in a pH 4 aqueous phase. The organic layer was separated and the aqueous layer was back-extracted with 2×150 mL=300 mL of ethyl acetate. The combined organic layers were washed with 2×54 mL=108 mL of 1.5% aqueous sodium sulfate prepared using deionized water, then 9.63 g (96.3 mmol) of potassium bicarbonate and 200 mL of deionized water were added. The resulting mixture was concentrated at 40° C./80-60 torr to give 215 g of an orange aqueous solution of 6 which was used directly in the next step.

### Example 6

### Preparation of Sulfone Acid Salt 7

A 500-mL, three-necked flask equipped with a mechanical stirrer, thermometer, pH probe, and a precision liquid addition pump was charged with the aqueous solution of 6 (215 g) from the previous step, which was calculated to contain 40.77 g (102 mmol) of 6. An additional 16 mL of deionized water was used to aid the complete transfer. Then, 1.84 g (5.58 mmol) of sodium tungstate dihydrate was added, followed by 150 mL of acetone. The pH of the solution was 7.1. Then, 1.69 g (16.9 mmol) of potassium bicarbonate was added and the mixture was stirred for 60 min to allow for pH equilibration. To the resulting murky, pH 7.82 solution was added 20.88 mL (204 mmol) of 30% hydrogen peroxide over 10 min at a constant rate. At the end of the addition, the temperature and pH of the mixture reached 30° C. and 7.46, respectively. The mixture was then stirred for 20 min without oxidant addition. To the resulting pH 7.55 solution was then added an additional 10.44 mL (102 mmol) of 30% hydrogen peroxide over 5 min. The pH decreased to 7.32 during the addition, then gradually increased to 8 over the course of 3 h. The pH was adjusted to 7.50 by the addition of 0.55 mL (9.57 mmol) of acetic acid and the mixture was stirred for 16 h. HPLC analysis indicated the presence of 7.6 area % of the sulfoxide intermediate. Thus, an additional 10.44 mL (102 mmol) of 30% hydrogen peroxide was added over 5 min, which lowered the pH from 7.75 to 7.5, and the reaction mixture was stirred for an additional 2.5 h. HPLC analysis indicated 1.35 area % of sulfoxide intermediate. Thus, 1.84 g (5.39 mmol) of sodium tungstate dihydrate was added, the pH was adjusted from 7.87 to 7.58 by the addition of 0.05 mL (0.87 mmol) of acetic acid and the reaction mixture was stirred for an additional 16 h. HPLC analysis indicated essentially complete reaction (0.51 area % of sulfoxide intermediate). Excess peroxide was quenched by the addition of 33.29 g (200 mmol) of potassium sulfite hydrate, while maintaining the temperature of the mixture below 40° C.. A starch/iodide paper test indicated complete quench. The mixture was then concentrated at 45° C./50 torr to remove organic solvents, and 150 mL of ethyl acetate was added, followed by 45 mL (145 mmol) of 50% aqueous citric acid solution. After thorough mixing of the two layers, the organic layer was separated and the aqueous layer (pH 5) was back-extracted with 250 mL of ethyl acetate. The combined organic layers were washed with 2×75 mL=150 mL of deionized water, and 13.18 mL (102 mmol) of racemic α-methylbenzylamine (rac-MBA) was added. The resulting mixture was stirred for 30 min, then concentrated at 45° C./70 torr to give a thick slurry, which was diluted with 450 mL of ethyl acetate and re-concentrated at 45° C./70 torr. The resulting thick slurry was diluted with 450 mL of acetonitrile and concentrated at 45° C./70 torr to give 68 g of a residue, which was diluted with 190 mL of acetonitrile. The suspension was heated briefly to reflux and, after cooling to ambient temperature, the solid was collected by filtration, washed with 75 mL of cold (4° C.) acetonitrile and dried by suction to give 47.76 g (84.6 % yield from 6) of the salt of 7 as a white solid 95.59% pure as determined by HPLC analysis.

### Example 7

### Preparation of Chiral Sodium Salt of 8

A 500-mL separatory fimel was charged with 250 mL of ethyl acetate, 44.82 g (81.2 mmol) of the salt of 7 obtained above and 300 mL of water. Then, 31.2 mL (81.2 mmol) of 50% aqueous citric acid was added and the two-phase mixture was thoroughly mixed. The organic layer was separated and the aqueous layer was back-extracted with 150 mL of ethyl acetate. The combined organic layers were washed with 2×150 mL=300 mL of water, followed by a solution of 0.68 g (8.1 mmol) of sodium bicarbonate in 250 mL of water (a small amount of brine was also added to facilitate phase separation), which improved the purity of 7 to 99.2% as determined by HPLC analysis.

Then, 8.20 g (82.8 mmol) of 97% sodium tert-butoxide was added in portions, while maintaining the temperature at ca. 16° C. with ice-water cooling. The mixture was diluted with 150 mL of heptane and concentrated at ca. 30° C./80 torr to a weight of ca. 103 g. This mixture was transferred to a 500-mL, three-necked flask (equipped with a magnetic stirrer, thermometer, distillation head and nitrogen gas inlet/bubbler) with the aid of 300 mL of heptane and 30 mL of ethyl acetate. The resulting mixture was concentrated by distillation at atmospheric pressure to a volume of ca. 250 mL. Then, while continuing the distillation, a total of 300 mL of ethanol was added. When the temperature of the mixture and distillate reached 77-80° C. and 77° C., respectively, the resulting concentrate (ca. 200 mL) was diluted with 100 mL of 1:1 ethanol:heptane, then partially concentrated by atmospheric distillation. An additional 100 mL of 1:1 ethanol:heptane was added and the mixture was re-concentrated by atmospheric distillation until the water content of the distillate reached 0.19 wt %, as determined by Karl-Fischer titration. To the resulting concentrate (ca. 90 g) was added 3.95 g (39.87 mmol) of 97% sodium tert-butoxide and 140 mL of ethanol. After removal of ca. 100 mL of solvent by atmospheric distillation, the resulting slurry was heated to reflux for 3 h, then allowed to cool to ambient temperature overnight. The resulting precipitate was collected by filtration, washed with 180 mL of 2:1 heptane:ethanol and dried by suction to give 25.16 g (68.4% yield) of the sodium salt of 8 as a white solid The chemical purity of this material was 98.1% (disregarding diastereomers) and the ratio of the desired diastereomer, (2R,3′R)-isomer 8, to the undesired isomers (2R,3′S and 2S,3′R, respectively) was 95.76:0.33:3.90 as determined by HPLC analyses. The mother liquor was transferred to a 250-mL three-necked flask (equipped with a magnetic stirrer, thermometer, distillation head and nitrogen gas inlet/bubbler) and concentrated by atmospheric distillation to a slurry (ca. 32 g), which was then heated to reflux for 3.5 h and allowed to cool to ambient temperature overnight. The solids were collected by filtration, washed with 30 mL of 2:1 heptane:ethanol and dried by suction to give 5.32 g (14.5% yield) of the sodium salt of 8 as a tan solid. HPLC analysis indicated a chemical purity of 97.0% and a diastereomeric ratio (2R,3′R:2R,3′S:2S,3′R) of 92.43:1.68:5.89 for this material. These two crops were separately subjected to the ketal deprotection described in Example 8.

### Example 8

### Preparation of Keto-Acid 9

A 500-mL flask equipped with a magnetic stirrer was charged with 25.16 g (55.55 mmol) of the sodium salt 8, 53 mL of acetone and 26 mL (78 mmol) of 3N hydrochloric acid. After stirring at ambient temperature for 5 h, the mixture was diluted with 227 mL of deionized water and stirred overnight. The resulting precipitate was collected by filtration, washed with 151 mL of deionized water and dried by suction to give 18.76 g (97.9% yield) of crude 9 as a white solid; 99.60% pure and 92.7% de as determined by HPLC analyses. A 250 mL flask equipped with a magnetic stirrer and reflux condenser was charged with 18.66 g (54.12 mmol) of the crude 9 prepared above and 47 mL of acetone. The suspension was heated to reflux for 3 h, diluted by the slow addition of 47 mL of heptane over a period of 15 min, and allowed to cool to ambient temperature. The precipitate was collected by filtration, washed with 30 mL of 1:1 heptane:acetone and dried by suction to give 17.54 g (92.1% yield from 8) of 9 as a white solid. HPLC analysis of this material indicated a chemical purity of 99.70 % and a diastereomeric ratio (2R,3′R:2S,3′R) of 99.45:0.55. The second crop of 8 from the previous step was converted to 9 of a similar purity (diastereomeric excess of 99.6%) in 53.4% yield

### Example 9

### Preparation of IPA Solvate 11

A 500-mL flask equipped with a magnetic stirrer, dropping funnel and nitrogen gas inlet/bubbler was charged with 19.44 g (56.4 mmol) of 9,155 mL of dichloromethane and 0.25 mL (3.23 mmol) of DMF. To the suspension was added 5.2 mL (59.6 mmol) of oxalyl chloride over 5 min and the mixture was stirred at 20-22° C. for 2 h until gas evolution ceased and a clear light yellow solution was obtained. This solution was partially concentrated at 20° C./70 torr (ca. 20 mL of solvent was removed) and was added to a cold mixture of 6.70 g (70.4 mmol) of aminopyrazine, 7.0 mL (86.5 mmol) of pyridine and 195 mL of dichloromethane over 22 min, while maintaining the temperature of the mixture at −16±6° C.. After stirring for an additional 1.5 h at that temperature, the reaction mixture was allowed to slowly warm to −5° C., then was quenched by the addition of 2.2 mL (122 mmol) of deionized water. After stirring for 20 min at −5° C. to 0° C., 49 g of silica gel 60 (230-400 mesh) was added and the stirred mixture was allowed to warm to ambient temperature over 1.5 h. The solids were removed by filtration and washed with 1.6 L of 1:1 ethyl acetate:dichloromethane. The combined filtrate and washes were concentrated under reduced pressure to a volume of ca. 500 mL, washed with 90 mL of 1N hydrochloric acid, 2×120 mL=240 mL of deionized water, 120 mL of 1M potassium bicarbonate and 140 mL of 0.3% aqueous sodium sulfate, and concentrated at 45° C. under reduced pressure. The resulting concentrated solution of 10 was diluted with 500 mL of ethyl acetate, concentrated at 45° C. under reduced pressure, diluted again with 500 mL of ethyl acetate and re-concentrated to remove residual water. The resulting residue was dissolved in 320 mL of 2-propanol and the solution was partially concentrated to remove ethyl acetate. Additional 2-propanol was added to adjust the volume to ca. 320 mL and the resulting mixture was heated to reflux to obtain a clear orange solution, then allowed to slowly cool to ambient temperature over 3 h. The resulting crystals were collected by filtration, washed with 71 mL of 2-propanol and dried by suction to give 22.04 g (81.1% yield) of 11 as a pale yellow solid HPLC analysis of this material indicated a chemical purity of 99.94% and a diastereomeric purity of 100%.

### Example 10

### Characterization of the Compound of Formula (I)

Various analytical techniques were used to characterize the IPA solvate and amorphous form:

### Powder X-Ray Diffractometry

The powder X-ray diffraction pattern was recorded with Scintag X-1 X-ray diffractometer. Sample was placed on zero background sample holder and scanned 2 theta range from 1 degree to 40 degree at scanning rate of 1 degree per minute at ambient conditions.

### Thermogravimetric Analysis

Thermogravimetric analysis on sample was performed using Perkin Elmer Pyris-1 TGA along with Nicolet Magna-IR Spectrometer Model 750 for off-gas analysis. Sample was loaded onto platinum pan and was heated at heating rate of 2° C./min or 10° C./min.

### Differential Scanning Calorimetry

Differential scanning calorimetry was performed using Perkin Elmer DSC-7 and Diamond DSC, using sealed aluminum pan with a hole in the lid. Sample was heated at a rate of 10° C./min.

### Vapor Sorption Analysis

The interaction of IPA solvate with water vapor was studied using a dynamic vapor sorption analyzer (VTI vapor sorption analyzer SCA-100 and MB-300G) at various temperatures.

### Particle Size Analysis

The particle size distribution of IPA solvate was measured by laser diffraction using Malvern Mastersizer 2000. Dispersing agent was 0.1% Span in hexane. The sample was sonicated for two minutes at 100% power prior to loading to the cell. The diffraction data was analyzed using the Fraunhofer model.

### Optical Microscope

Morphology of drug particle was examined using a Leitz Aristomet optical microscope or Hi-Scope KH-3000 for high magnification examination.

Comparative Characterization of IPA Solvate and Amorphous Form

### Single Crystal X-Ray Crystallography

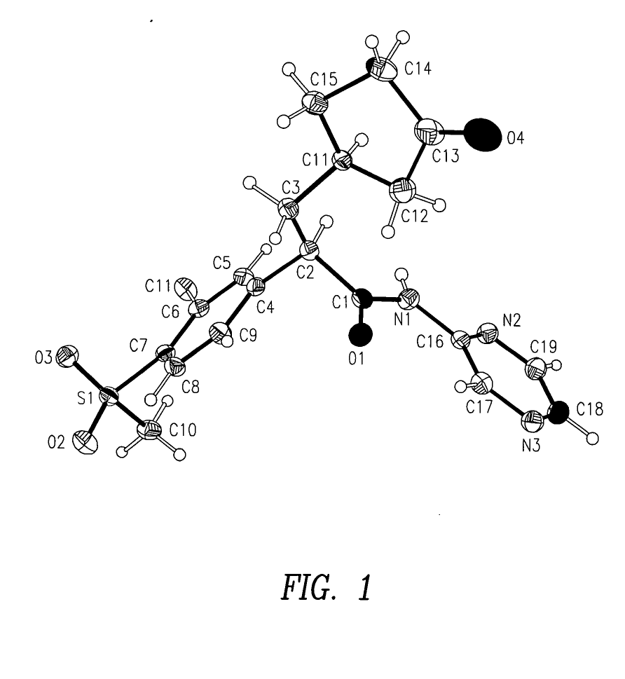

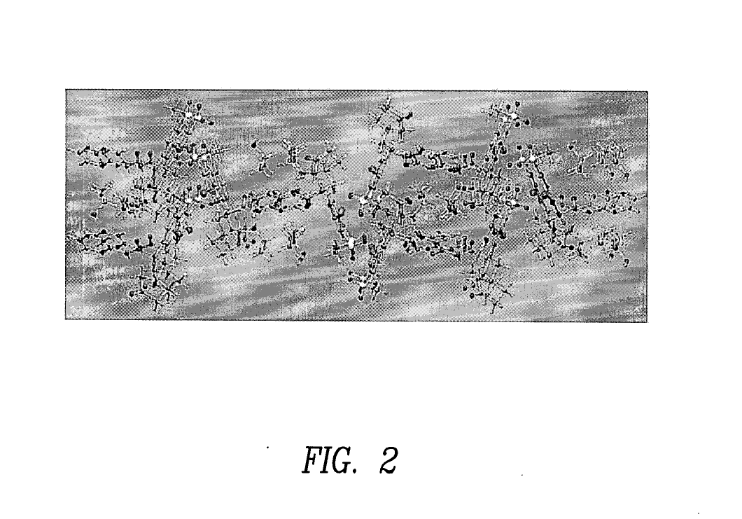

Single crystal structure analysis of the compound of formula (I) was performed by the X-ray Lab of Johns Hopkins University, Baltimore, Md. It confirmed the chemical structure with (R,R) configuration. FIGS. 1 and 2.

### Photomicrographs

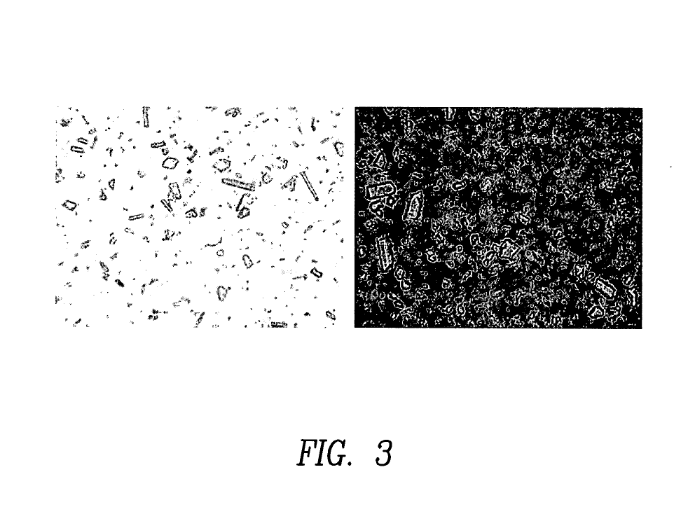

The un-milled IPA solvate had rod and plate-like crystal morphologies with varying particle size distribution. The photomicrographs of typical IPA solvate are shown in FIG. 3.

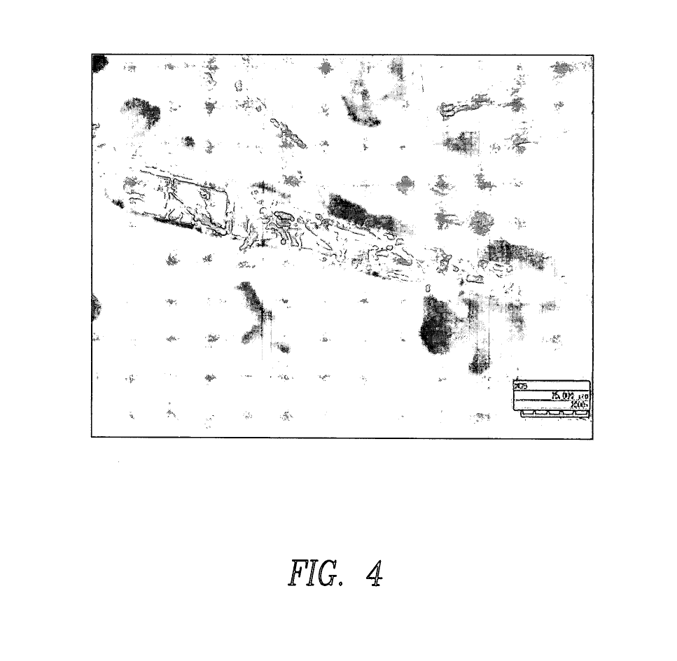

When viewed under the Hi-Scope at higher magnification (2000×), smaller particles appears to be attached to the larger particles, as seen in FIG. 4.

### Particle Size Determination

The particle size distribution of IPA solvate was measured using a Malvern Mastersizer 2000 using 0.1% SPAN in hexane as dispersing medium. As observed under microscope, the un-milled IPA solvate had wide range of particle size distribution, which varied from lot to lot. (Table 1)

TABLE 1
Particle Size Distribution of IPA Solvate

| | Particle size (μm) | | |
|---|---|---|---|
| Lot No. | D (0.1) | D (0.5) | D (0.9) |
| 1 | 2.3 | 12.6 | 34.7 |
| | 2.2 | 11.8 | 34.2 |
| | 2.2 | 11.5 | 33.8 |
| 2 | 3.1 | 51.5 | 162.8 |
| | 3.2 | 50.9 | 160.7 |
| | 3.2 | 49.9 | 157.6 |
| 3 | 6.4 | 86.6 | 451.1 |
| | 6.3 | 87.0 | 388.2 |
| | 6.6 | 88.9 | 328.1 |

### Powder X-Ray Diffraction Pattern

The powder XRD pattern of the compound of formula (D was examined by powder X-ray diffraction under ambient conditions. All batches of IPA solvate evaluated yielded one powder X-ray pattern (Table 2).

TABLE 2
List of Batches Examined

| Lot Number | Result |
|---|---|
| 1 | Form I |
| 2 | Form I |
| 3 | Form I |
| 4 | Form I |
| 5 | Form I |
| 6 | Form I |
| 7 | Form I |
| 8 | Form I |
| 9 | Form I |
| 10 | Form I |
| 11 | Form I |
| 12 | Form I |
| 13 | Form I |
| 14 | Form I |
| 15 | Form I |
| 16 | Form I |

The IPA solvate had specific diffraction peaks attributable to the crystalline lattice. The experimentally obtained two theta peaks were in good agreement with the calculated values from single X-ray crystallography data. (Table 3)

TABLE 3
Powder XRD Peak of the Compound of Formula (I)

| | | Experimental |
|---|---|---|
| Peak ID | Calculated | (Lot 2) |
| 1 | 6.32 | 6.26 |
| 2 | 9.98 | 9.88 |
| 3 | 12.54 | 12.59 |
| 4 | 15.72 | 15.70 |
| 5 | 16.68 | 16.58 |
| 6 | 17.26 | 17.29 |
| 7 | 17.62 | 17.93 |
| 8 | 19.92 | 19.84 |
| 9 | 20.56 | 20.20 |
| 10 | 21.46 | 21.24 |
| 11 | 23.94 | Not Detected |
| 12 | 24.40 | 24.46 |
| 13 | Not Detected | 24.90 |

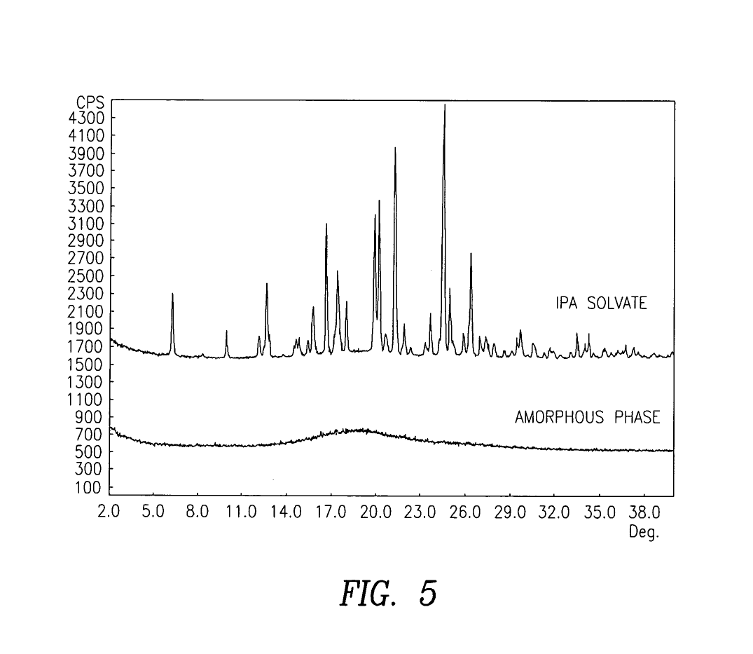

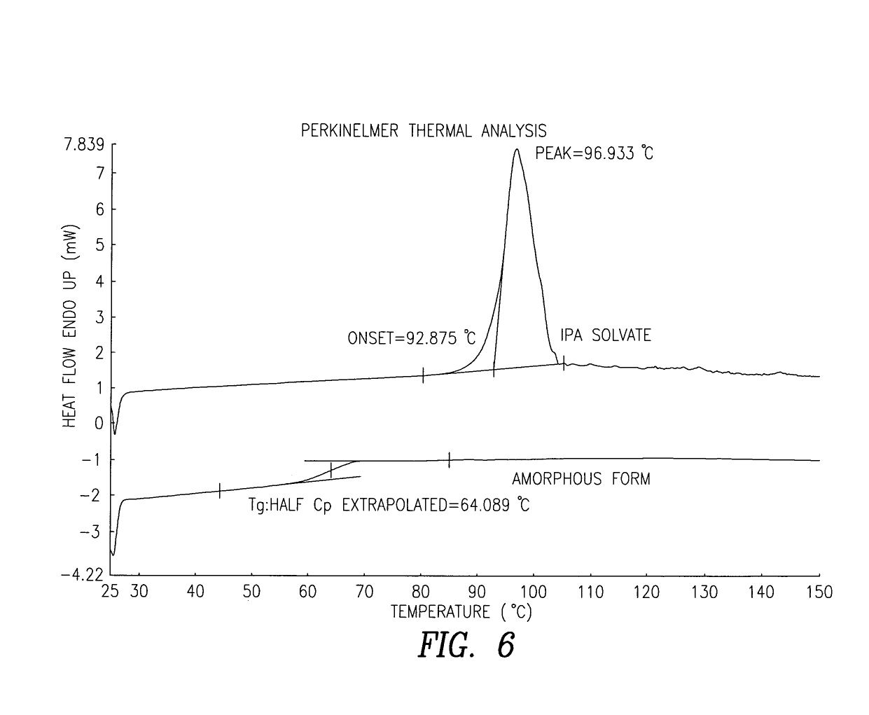

By contrast, the amorphous form showed a halo pattern and exhibited no specific diffraction peak, which is characteristic of an amorphous form. (FIG. 5)

### Thermal Analysis by Differential Scanning Calorimetry (DSC)

The IPA solvate underwent a melt transition around 94° C. accompanied by desolvation of isopropyl alcohol. No endothermic event for solvent loss was observed prior to the melt transition. The ΔH for this thermal transition was measured to be 111.3 J/g (46.96 KJ/mole) (Table 4).

TABLE 4
Heat of Transition of IPA Solvate (n = 7)

| Lot No | ΔH, J/g |
|---|---|
| 2 | 117.336 |
| 2 | 114.903 |
| 2 | 116.268 |
| 2 | 107.334 |
| 2 | 104.215 |
| 2 | 109.873 |
| 2 | 109.190 |
| Mean | 111.303 |
| St Dev | 4.942 |
| RSD | 4.44% |

In comparison, the amorphous compound exhibited no endothermic melt transition; however, a glass transition temperature was observed around 60-74° C. FIG. 6 shows the DSC thermogram of IPA solvate and amorphous form.

### Thermal Analysis by Thermogravimetric Analysis (TGA)

The IPA solvate placed onto platinum sample holder was heated from 25° C. to 300° C. at 10° C. per minute under nitrogen flow. No weight loss (solvent loss) was observed prior to the melt transition of IPA solvate. The weight loss after melt for two lots was about 12.58 wt % and 12.71 wt %, which corresponded to the theoretical weight loss of the mono isopropyl alcohol solvate (12.47%), within experimental error.

In comparison, the amorphous compound showed non specific weight loss of about 1-2% which can be attributed to the loss of residual solvents. (FIG. 7)

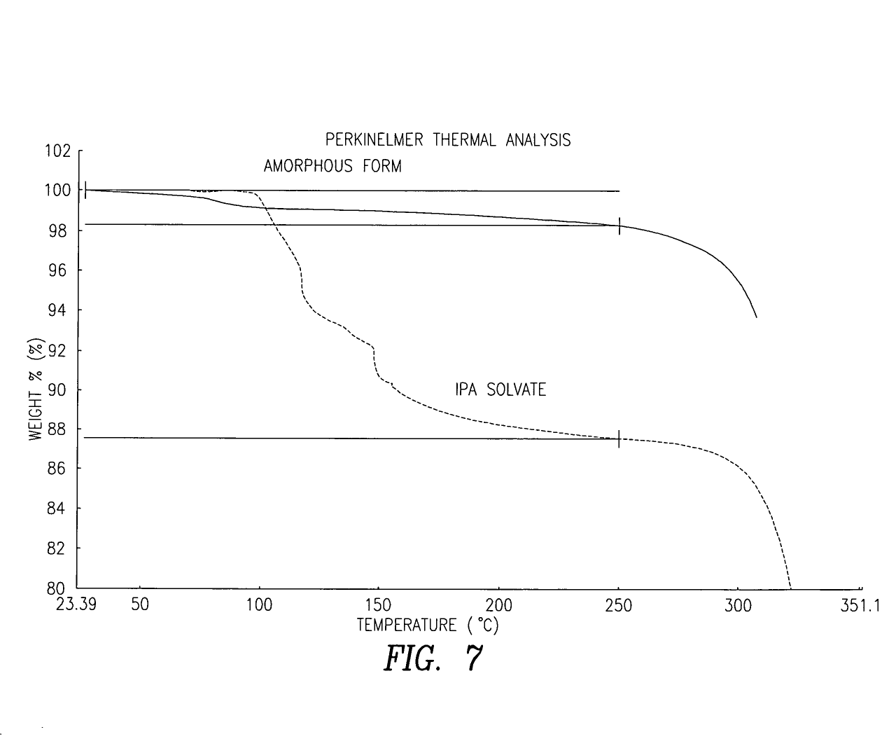

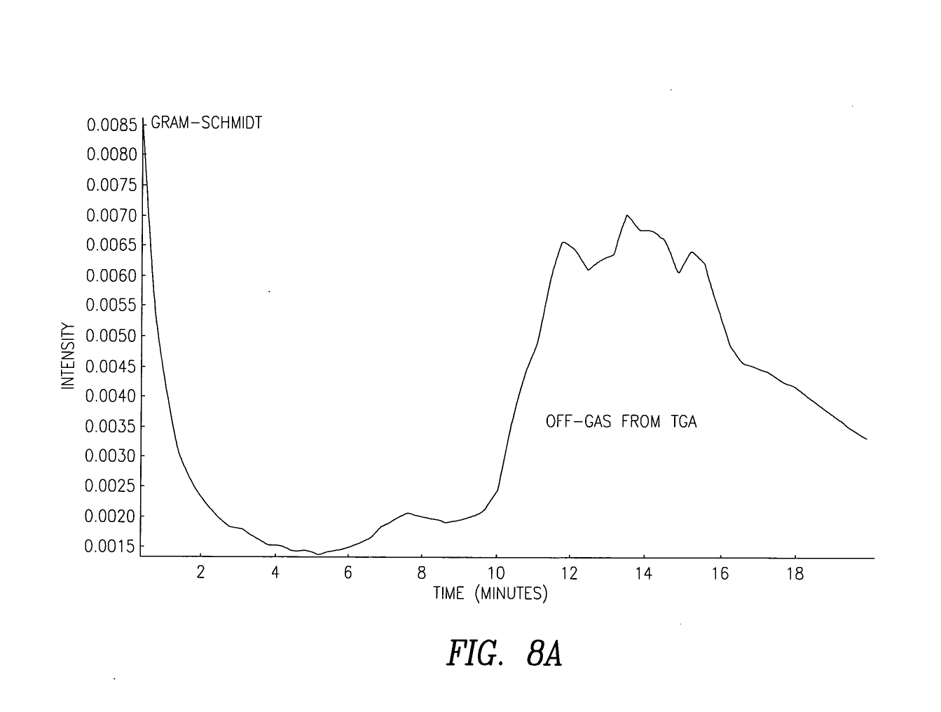

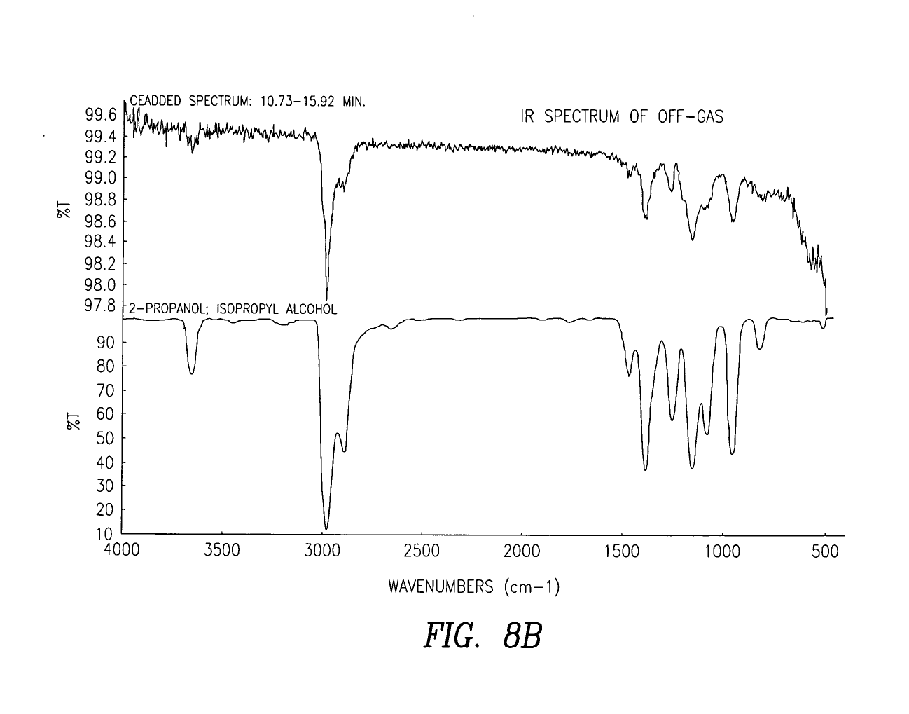

The gas evolved from IPA solvate upon desolvation was analyzed by TGA-IR. It was confirmed that the evolved gas was isopropyl alcohol. (FIG. 8)

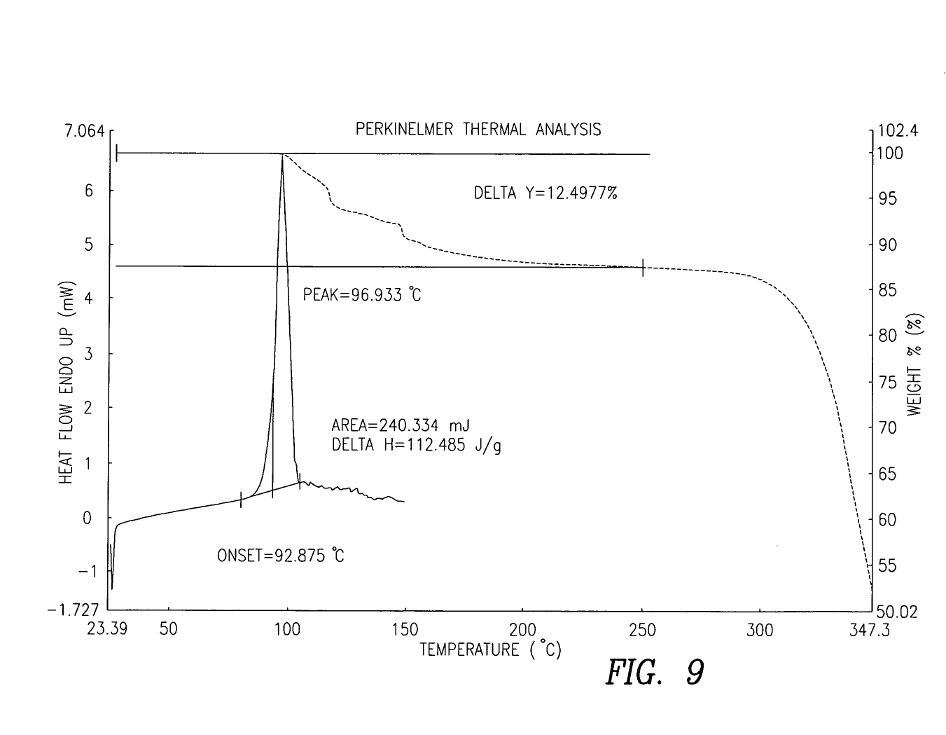

The overlaid DSC and TGA thermogram of IPA solvate are shown in FIG. 9, where weight loss of IPA solvate is associated with melt transition.

The weight loss of IPA solvate by TGA was reproducible as seen in FIG. 10, where 3 runs of same sample were overlaid. The mean weight loss was 12.71±0.015% from 25 to 260° C.. Interestingly, it was noted that IPA evolution was extended over the wide range of temperature in a non-continuous fashion.

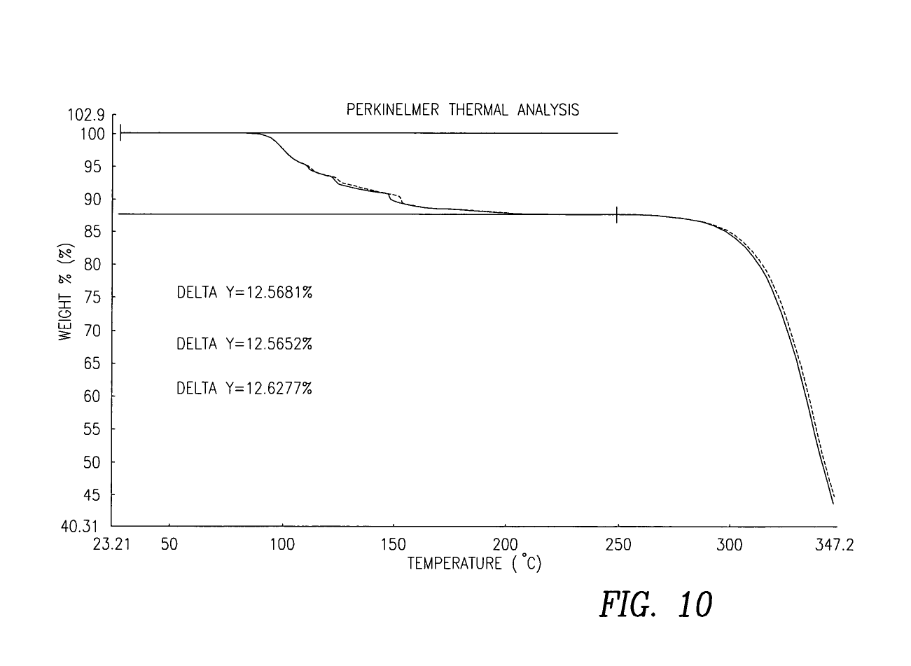

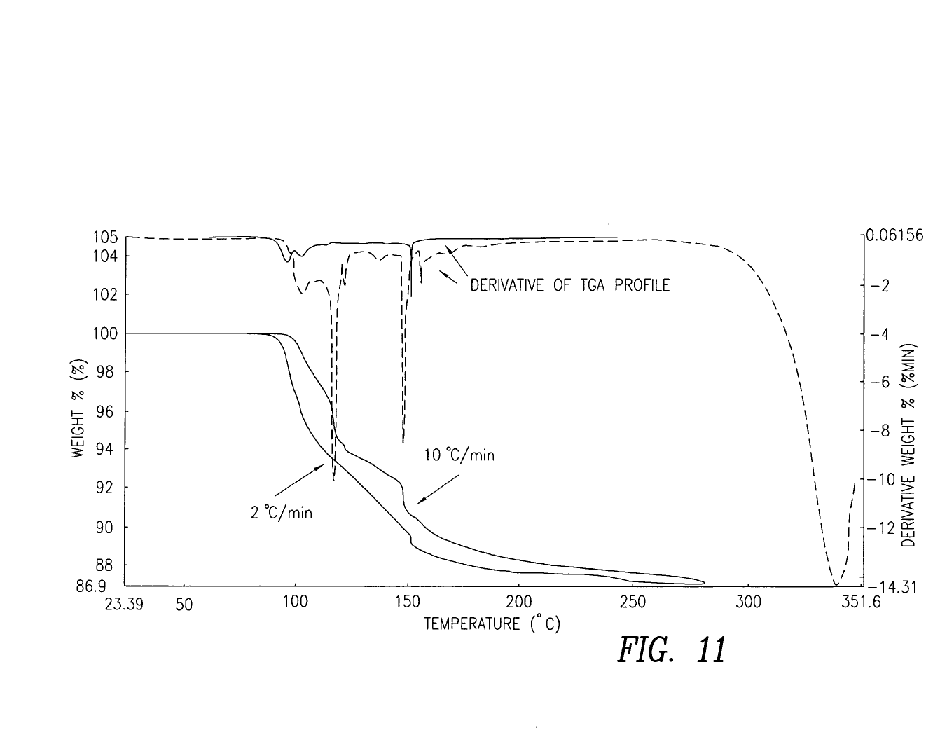

Heating with a slower ramp yielded a similar profile, suggesting that the loss of isopropyl alcohol from the melt was not due to kinetics of evaporation alone. The derivatives of TGA weight loss confirmed non-continuous loss of isopropyl alcohol, the profile of which varied depending on the heating ramp. FIG. 11 shows the TGA thermogram of IPA solvate.

### Moisture Sorption Analysis

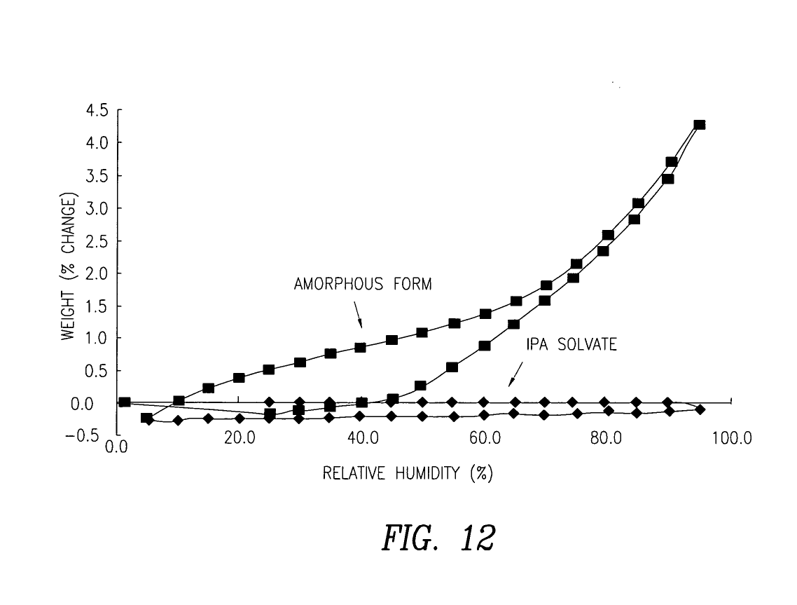

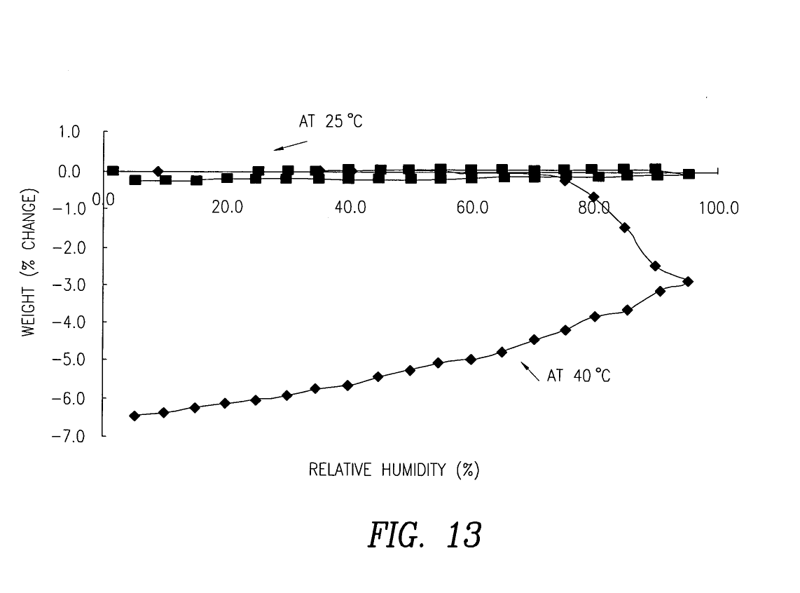

The IPA solvate was non hygroscopic with minimal weight changes from ambient humidity to 95% RH at 25° C. (FIG. 12). When a moisture sorption study was conducted at 40° C., the weight loss as a result of interaction with water vapor was resulted as seen in FIG. 13.

As expected, the IPA solvate with different particle size distribution showed different onset of critical relative humidity, in which the IPA solvate began to interact with moisture.

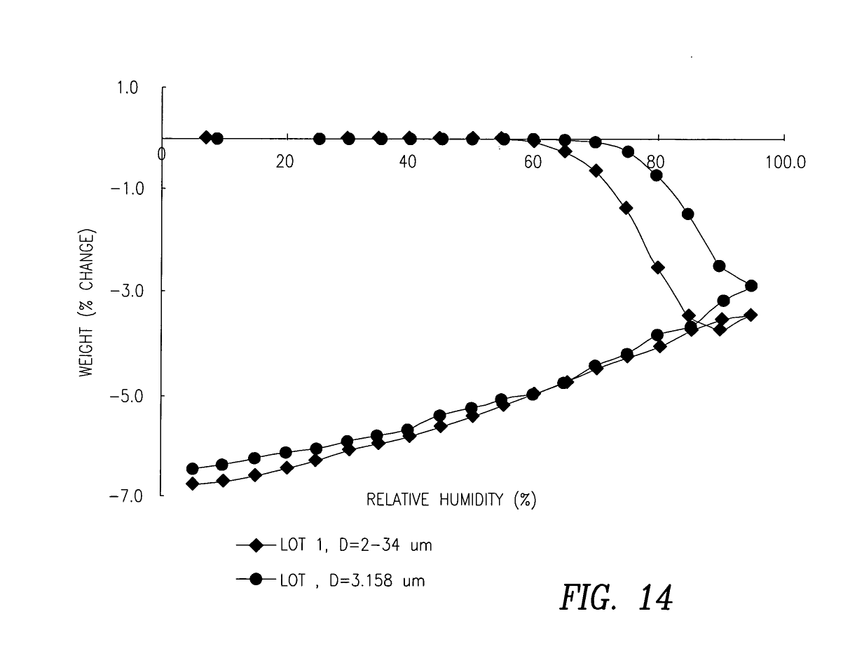

FIG. 14 shows moisture sorption isotherms of IPA solvate with different particle size distribution. These results suggest that interaction of IPA solvate with water vapor will depend on particle size of the sample, whereby the smaller the particle size, the lower the water vapor pressure. That is, the sample with the smaller particle will have an increased number of contact points, which in turn can result in capillary condensation at lower water vapor pressure.

### Evaluation of Crystallinity of IPA Solvate by DSC

The heat of fusion is the change in enthalpy for the conversion of a solid to a liquid at constant pressure and temperature. It is the energy required to break down the bonds of molecules in the crystalline lattice.

Upon heating, the IPA solvate underwent a melting transition accompanied by desolvation process. It occurred with the onset temperature around 94° C. and peaked at 98° C.. The amorphous drug did not exhibit these thermal events, rather a glass transition temperature at around 60-70° C.

It was noticed that the heat of fusion of IPA solvate varied from lot to lot presumably due to different quality of crystals. For example, one yielded 111.30±4.94 J/g while another lot yielded 107.052±1.80. Lower heat of fusion can be attributed to imperfection of crystals. The apparent heat of fusion of IPA solvate was used to estimate the crystallinity of IPA solvate.

The crystalline IPA solvate and amorphous drug substance were thoroughly mixed using a Wig-L-Bug for 20 seconds with 2 sapphire beads at ratios of 25%, 50%, and 75% (w/w). The crystallinity of IPA solvate was assumed to be 100% while the crystallinity of amorphous drug substance was assumed to be 0% in this experiment. It was confirmed that milling did not cause any polymorphic changes.

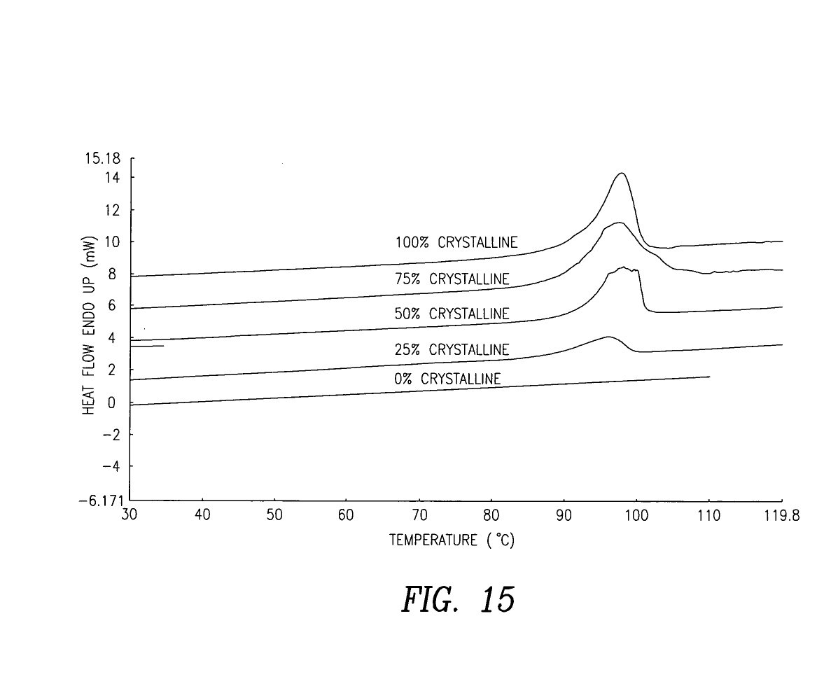

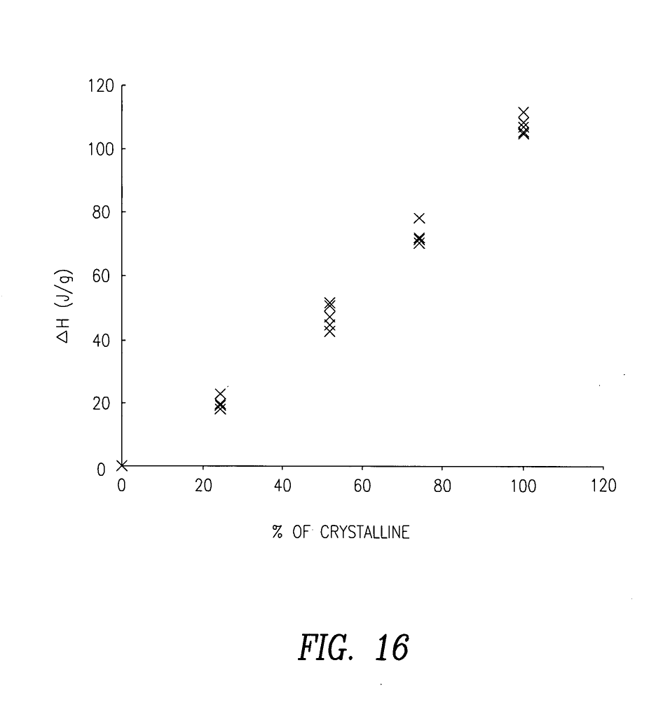

Typical DSC thermograms of calibration mixtures are shown in FIG. 15. The mean heat of fusion data from the calibration mixtures are listed in Table 5 and the plot is shown in FIG. 16. The heat of fusion had a linear relationship to crystallinity with a correlation coefficient better than 0.98.

TABLE 5
Heat of Fusion at Various Mixing Ratio (n = 5)

| Ratio of IPA Solvate and | % | | |
|---|---|---|---|
| Amorphous | Crystallinity | ΔH (J/g) | STD |
| 100% Amorphous | 0.0% | 0.000 | N/A |
| 75:25 Amorphous:IPA solvate | 24.5% | 19.826 | 2.73 |
| 50:50 Amorphous:IPA Solvate | 51.8% | 47.118 | 3.73 |
| 25:75 Amorphous:IPA Solvate | 74.3% | 72.487 | 2.97 |
| 100% IPA Solvate | 100.0% | 107.052 | 1.80 |

### Relationship Between % IPA Level and Heat of Fusion of Drug Substance

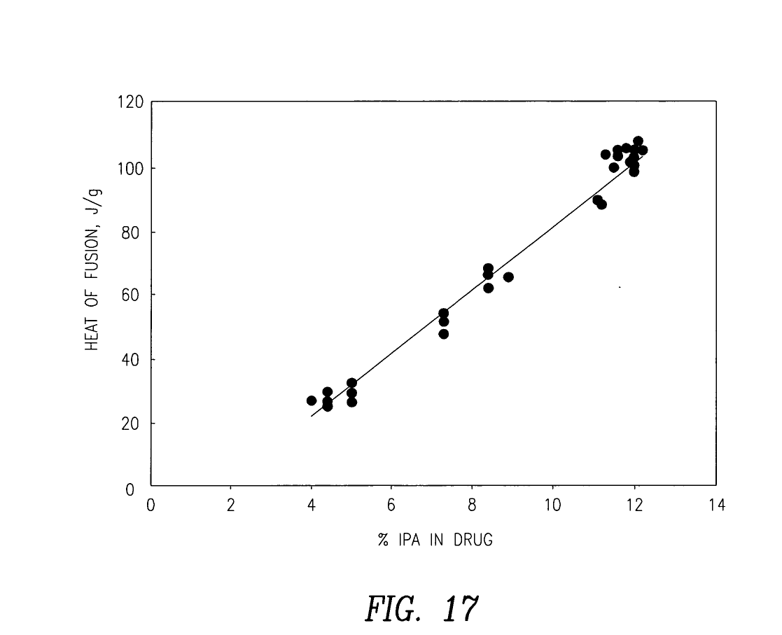

The stability sample obtained at various time points and conditions were analyzed for the IPA level and heat of fusion (Table 6). These two variables were plotted against each other to see the correlation (FIG. 17). Strong correlation between these parameters was observed with R value of 0.9898.

TABLE 6
List of Stability Samples Analyzed for IPA Content and Heat
of Fusion

| Lot | Storage condition | Time (month) | IPA (% w/w) | ΔH (J/g) |
|---|---|---|---|---|
| 1 | 40° C./75% RH/Fiber | 1 | 8.9 | 65.084 |
| | | 3 | 4.4 | 29.404 |
| | | | 4.4 | 26.416 |
| | | | 4.4 | 24.713 |
| | | | 4.4 | 28.733 |
| | | 6 | 4 | 26.529 |
| | 40° C./75% RH/Metal | 1 | 11.1 | 89.35 |
| | | 3 | 7.3 | 53.712 |
| | | | 7.3 | 51.302 |
| | | | 7.3 | 47.200 |
| | | 6 | 5 | 32.201 |
| | | | 5 | 29.011 |
| | | | 5 | 26.128 |
| | 25° C./60% RH/Fiber | 3 | 12 | 99.861 |
| | | 6 | 12 | 98.112 |
| | 25° C./60% RH/Metal | 3 | 12 | 102.665 |
| | | 6 | 11.9 | 101.346 |
| | 50° C./Metal | 1 | 12 | 102.033 |
| | | | 12 | 99.554 |
| | 50° C./Fiber | 1 | 11.8 | 105.445 |
| | 5° C./Fiber | 6 | 12.2 | 104.917 |
| | 30° C./60% RH/Metal | 10 | 11.5 | 99.538 |
| | 30° C./60% RH/Fiber | 10 | 11.2 | 87.929 |
| 3 | 40° C./75% RH/Metal | 1 | 11.6 | 105.006 |
| | | | 11.6 | 102.972 |
| | | 3 | 11.3 | 103.326 |
| | | | 11.3 | 103.52 |
| | | 6 | 8.4 | 65.867 |
| | | | 8.4 | 67.851 |
| | | | 8.4 | 61.796 |
| | 25° C./60% RH/Metal | 3 | 12 | 104.847 |
| | | 6 | 12 | 102.406 |
| | 5° C./Metal | 3 | 12.1 | 107.801 |
| | | 6 | 12.1 | 106.157 |
| | 50° C./Metal | 1 | 12 | 100.639 |

Sample was stored in a double PE bag with either metal drum or fiber drum as the container.

### Evaluation of Crystallinity of IPA Solvate after Exposure to High Humidity

In order to study if water vapor can replace isopropyl alcohol in the crystalline lattice and form a hydrate, the following study was conducted.

About 20 mg of IPA solvate was placed in a VTI moisture sorption analyzer set at 95% relative humidity and 25° C. for 4 days. After 4 days in this condition, the IPA solvate lost about 3.8% weight.

Along with the stability sample at 40° C./75% RH for 3 months, this water vapor treated sample and a control sample were examined by powder X-ray diffraction to observe any pattern changes.

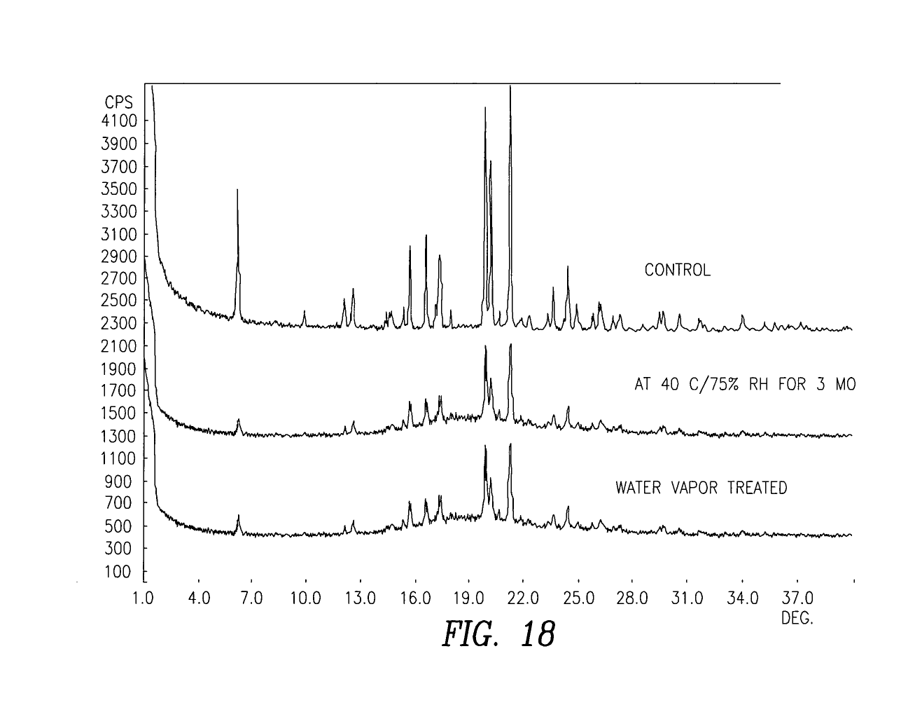

FIG. 18 shows the powder XRD pattern of water vapor treated IPA solvate.

Other than overall reduction of peak intensity, no changes in powder XRD pattern were observed.

It is to be understood that the invention is not limited to the particular embodiments of the invention described above, as variations of the particular embodiments may be made and still fall within the scope of the appended claims.

What is claimed is:

1. A crystalline isopropanol solvate of 2(R)-(3-Chloro-4-methanesulfonyl-phenyl)-3-((R)-3-oxo-cyclopentyl)-N-pyrazin-2-yl-propionamide of the formula (I):

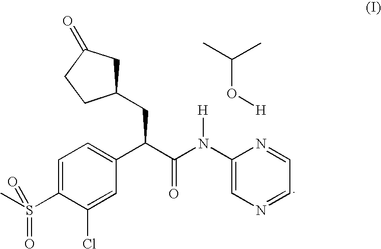

2. A pharmaceutical composition, comprising a therapeutically effective amount of 2(R)-(3-Chloro-4-methanesulfonyl-phenyl)-3-((R)-3-oxo-cyclopentyl)-N-pyrazin-2-yl-propionamide, and a pharmaceutically acceptable carrier.

3. A method for the treatment of a metabolic disease or disorder, comprising administering a therapeutically effective amount of 2(R)-(3-Chloro-4-methanesulfonyl-phenyl)-3-((R)-3-oxo-cyclopentyl)-N-pyrazin-2-yl-propionamide to a patient in need thereof.

4. The method according to claim 3, wherein said metabolic disease or disorder is type II diabetes.
